(19) United States
(12) Patent Application Publication
(10) Pub. No.: US 2026/0259585 A1
(43) Pub. Date: Sep. 3, 2026

(54) CARBON FIBER-EMBEDDED WEARABLE DEVICES

(71) Applicant: Oura Health Oy, Oulu (FI)

(72) Inventors: Michael Chapp, Lafayette, CA (US); Jonathan Sami Watson, San Diego, CA (US); Jose Julio Doval, Escondido, CA (US); Jukka Tapani Mäkinen, Oulu (FI); Daniel Alonso-Soski, San Diego, CA (US); Matias Negatu, San Diego, CA (US); Christopher Walck, San Diego, CA (US); Christopher Popp, San Diego, CA (US); Hanna Hart, San Diego, CA (US); Chetan Bangalore Chikkamariyappa, Dublin, CA (US)

(21) Appl. No.: 19/467,590

(22) Filed: Feb. 2, 2026

Related U.S. Application Data
(60) Provisional application No. 63/753,824, filed on Feb. 4, 2025.

(51) Int. Cl. G06F 1/18; G06F 1/16; G06F 3/01; G06F 3/044; H05K 5/00; H05K 5/04

(52) U.S. Cl. CPC G06F 1/182; G06F 1/163; G06F 1/1635; G06F 1/1692; G06F 1/1698; G06F 1/189; G06F 3/011; G06F 3/044; H05K 5/0091; H05K 5/04; G06F 2203/04103

(57) ABSTRACT

Methods, systems, and devices for wearable ring devices formed via carbon fiber components are described. For example, a wearable ring device may include a ring-shaped housing including one or more carbon fiber layers, where electronic components of the wearable ring device (e.g., light-emitting components, light-receiving components, radio frequency (RF) transceiver components) are embedded within, or otherwise attached to, the one or more carbon fiber layers of the ring-shaped housing. For example, conductive traces and/or optical fibers may be embedded within (e.g., woven into) the carbon fiber layers of the ring-shaped housing.

### CROSS REFERENCE

The present Application for Patent claims the benefit of U.S. Provisional Patent Application No. 63/753,824 by CHAPP et al., entitled “CARBON FIBER-EMBEDDED WEARABLE DEVICES,” filed Feb. 4, 2025, assigned to the assignee hereof, and expressly incorporated by reference herein.

### FIELD OF TECHNOLOGY

The following relates to wearable devices and data processing, including wearable devices with carbon fiber components.

### BACKGROUND

Some wearable devices may be configured to collect data from users associated with the user’s heart rate, respiratory rate, and the like. However, in order to manufacture wearable devices that are comfortable for the user to wear for prolonged periods, the wearable devices must be manufactured to be extremely lightweight and thin, which may be particularly difficult in the context of small wearable devices, such as finger-worn wearable ring devices. As such, the manufacturing process for wearable devices may be extremely complex and expensive, which may increase the cost of the wearable devices.

### BRIEF DESCRIPTION OF THE DRAWINGS

FIG. 1 shows an example of a wearable device diagram illustrating a wearable ring device with carbon fiber components in accordance with aspects of the present disclosure.

FIG. 2 shows an example of a manufacturing diagram that supports wearable devices with carbon fiber components in accordance with aspects of the present disclosure.

FIGS. 3 and 4 illustrate examples of systems that support wearable devices with carbon fiber components in accordance with aspects of the present disclosure.

FIG. 5 shows a flowchart illustrating methods that support wearable devices with carbon fiber components in accordance with aspects of the present disclosure.

### DETAILED DESCRIPTION

Wearable devices have emerged as powerful tools for monitoring physiological parameters and providing health insights. However, the design and functionality of such wearable devices face significant limitations due to material constraints. Traditional wearable devices typically employ rigid materials and complex electronic component integration methods that result in bulky form factors, reduced user comfort, and manufacturing challenges. For example, in order to manufacture wearable devices that are comfortable for the user to wear for prolonged periods, the wearable devices must be manufactured to be extremely lightweight and thin, which may be particularly difficult in the context of small wearable devices, such as finger-worn wearable ring devices. As such, the manufacturing process for wearable devices may be extremely complex and expensive, which may increase the cost of the wearable devices. While various approaches have attempted to address these limitations through flexible printed circuit boards (PCBs) and alternative materials, these solutions often fail to provide the optimal balance of durability, functionality, and user comfort required for continuous health monitoring applications.

Accordingly, aspects of the present disclosure are directed to wearable devices that are manufactured (at least partially) from carbon fiber materials, such as carbon fiber reinforced plastic (CFRP) materials. The use of carbon fiber materials may enable the creation of lightweight, durable, and flexible wearable devices that provide improved user comfort.

Carbon fiber materials, such as CFRP materials, represent a class of advanced composites that include high-strength carbon fibers embedded within a polymer matrix. These materials exhibit exceptional strength-to-weight ratios, with carbon fibers typically measuring 5–10 micrometers in diameter and exhibiting tensile strength up to five times that of steel, while maintaining significantly lower mass. The polymer matrix (which may include epoxy resin), may serve to bind the fibers together, facilitate load transfer, and protect the fibers from environmental factors.

The integration of CFRPs within wearable ring devices may help address many of the limitations of conventional wearable devices. The unique properties of carbon fiber materials (including their high strength, low weight, and potential for electrical conductivity) make them particularly suitable for creating advanced wearable form factors. When properly engineered, CFRP structures may be used to accommodate embedded electronic components while maintaining structural integrity and providing additional functionalities, such as electromagnetic shielding or thermal management. Moreover, the manufacturing flexibility of CFRPs allows for the creation of complex geometries and the incorporation of multiple functional layers, enabling new approaches to sensor integration and power management in wearable devices. For example, as will be further described herein, different carbon fiber layers of a wearable ring device may be manufactured with different characteristics (e.g., varying rigidity/flexibility), which may improve the functionality and comfortability of the wearable ring device.

Despite the potential advantages of carbon fiber materials in wearable technology, previous wearable device solutions have not fully leveraged the capabilities of such materials for creating integrated, multi-functional device structures. Previous attempts to incorporate carbon fiber in wearable devices have typically focused on the mechanical properties of carbon fiber alone (e.g., reducing weight), and have failed to exploit the potential for electronic component integration, sensor housing, and power management. Accordingly, aspects of the present disclosure are directed to wearable ring devices made from carbon fiber materials (e.g., carbon fiber layers) in order to fully utilize the unique properties of the carbon fiber materials, thereby enabling more capable, comfortable, and efficient wearable ring devices.

In some aspects of the present disclosure, a housing of a wearable ring device (e.g., a ring-shaped housing) may be manufactured via one or more carbon fiber layers (e.g., CFRP layers), where electronic components (e.g., light-emitting components, light-receiving components, temperature sensors, other physiological sensors, radio frequency transceiver components, etc.) are embedded within, or otherwise attached to, the one or more carbon fiber layers of the ring-shaped housing. For example, conductive traces and/or optical fibers may be embedded within (e.g., woven into) the carbon fiber layers of the ring-shaped housing. That is, the carbon fiber structures may be interwoven with a “network” of conductive traces, conductive vias, optical fibers, or any combination thereof. In this regard, the carbon fiber material of the carbon fiber layers may help provide both the structural support and framework of the wearable device, as well as facilitate electrical connection and sensing capabilities throughout the device. A battery may power these components, while processors of the wearable ring device may manage the acquisition of physiological data through the light-based sensors and facilitate data transmission to a user device via the radio frequency components. By utilizing carbon fiber, aspects of the present disclosure may enable wearable devices with improved electrical conductivity and the potential for innovative features such as capacitive touch sensing and inductive charging, thereby addressing the shortcomings of existing technology and providing a more efficient and user-friendly wearable devices.

Techniques described herein may enable smaller, lighter, and more compact wearable devices. In particular, techniques described herein may enable electronic components of a wearable device to be woven directly into the carbon fiber layers of the wearable device, which may reduce (or eliminate) the need for a PCB within the wearable device, thereby reducing weight and thickness. Further, using CFRP materials may reduce the complexity and cost of the manufacturing process for wearable devices, thereby reducing the cost of the wearable devices themselves.

In this regard, aspects of the present disclosure leverage the unique properties of carbon fiber materials to create more capable and efficient wearable devices. The carbon fiber structure serves multiple functions (e.g., providing mechanical support, enabling component integration, and facilitating electrical connections) while maintaining a compact, lightweight form factor. This approach reduces manufacturing complexity while improving device durability and functionality. The solution offers several advantages, including enhanced sensor integration, improved power efficiency, and better user comfort through optimized material properties. Additionally, the carbon fiber structure enables advanced features such as integrated antennas and electrodes, while its layered construction provides flexibility for component placement and protection. These improvements result in a more sophisticated and user-friendly wearable device that overcomes the limitations of traditional approaches.

Aspects of the disclosure are initially described in the context of example wearable device diagrams. Additional aspects of the disclosure are described in the context of systems supporting physiological data collection from users via wearable devices. Aspects of the disclosure are further illustrated by and described with reference to apparatus diagrams, system diagrams, and flowcharts that relate to wearable devices with carbon fiber components.

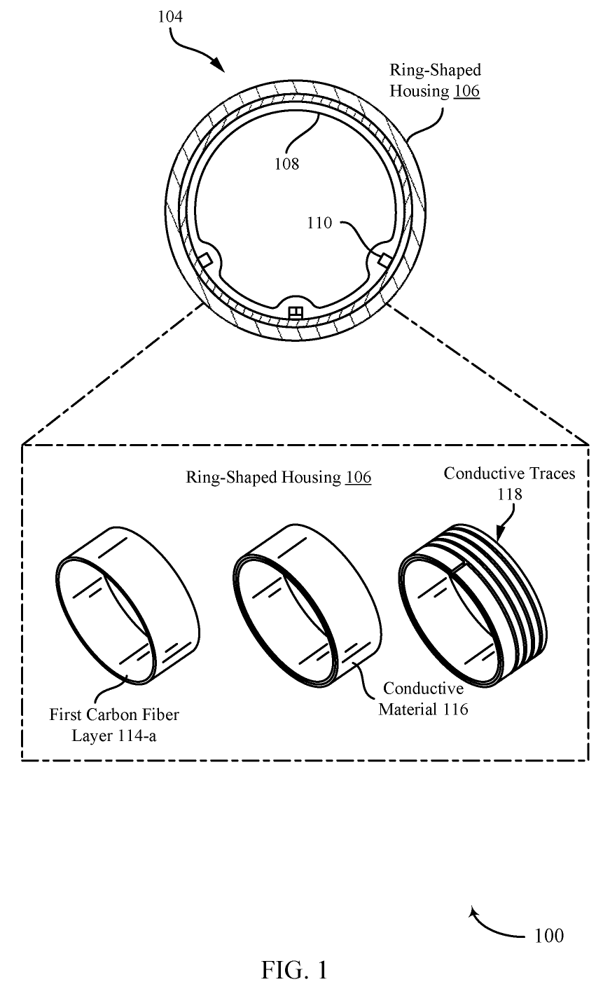

FIG. 1 shows an example of a wearable device diagram 100 that supports a wearable device with carbon fiber components in accordance with aspects of the present disclosure. For example, the wearable device diagram 100 illustrates a wearable ring device 104 manufactured (at least partially) using carbon fiber materials, according to aspects of the present disclosure.

The wearable ring device 104 may include a ring-shaped housing 106 configured to at least partially surround a finger of a user. The ring-shaped housing 106 may include one or more carbon fiber layers 114, and may be formed/designed to accommodate and protect various electronic components 110 that may be embedded within or attached to the carbon fiber layers 114.

The electronic components 110 may be configured to acquire physiological data from the user, such as heart rate information, heart rate variability (HRV) information, respiratory rate information, blood oxygen saturation information, skin temperature information, electrocardiogram (ECG) information, movement/activity data, sleep staging information, and the like. In this regard, the one or more electronic components 110 may include light-emitting components (e.g., light-emitting diodes (LEDs) and/or laser diodes), light-receiving components (e.g., photodetectors), temperature sensors, motion sensors (e.g., accelerometers, gyroscopes, inertial measurement units (IMUs)), radio frequency transceiver components (e.g., Bluetooth chips), and the like.

In some implementations, the ring-shaped housing 106 may include or define at least a portion of an outer curved surface (e.g., outer circumferential surface) and/or an inner curved surface (e.g., inner circumferential surface) of the wearable ring device 104 (where the inner curved surface is configured to at least partially contact the tissue of the user when the wearable ring device 104 is being worn by the user). In some implementations, the ring-shaped housing 106 may be constructed of one or more materials, such as carbon fiber materials (e.g., CFRP materials), molded materials (e.g., epoxy), and the like.

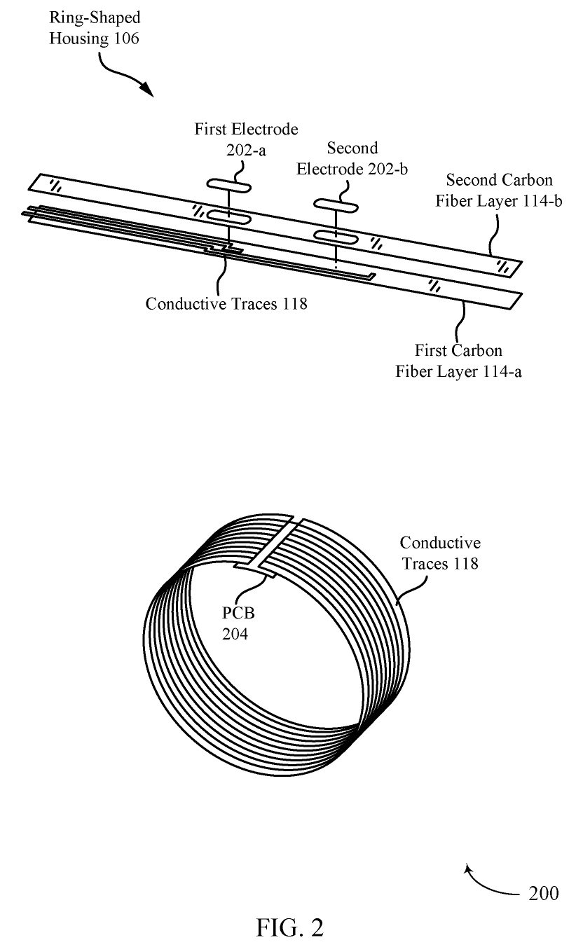

For example, as shown in FIG. 2, the ring-shaped housing 106 may be constructed from one or more carbon fiber layers 114 and a molded material 108. In such cases, the molded material 108 may include a transparent (or substantially transparent) epoxy material that is molded over the electronic components 110 of the wearable ring device. In this example, the one or more carbon fiber layers 114 may form the outer curved surface of the wearable ring device 104, where the molded material 108 may form the inner curved surface of the wearable ring device 104. In other cases, the ring-shaped housing 106 may be made of mostly (or entirely) carbon fiber layers 114 such that the carbon fiber layers form both the inner and outer curved surfaces of the wearable ring device 104. In other cases, a material (e.g., molded material) may be molded or otherwise disposed around the outer surface of the carbon fiber layers 114, such that the material forms the outer curved surface of the wearable ring device 104.

Carbon fiber materials, as implemented in the wearable ring device 104, may provide unique advantages through their layered composite structure and customizable properties. The carbon fibers, typically arranged in precisely oriented layers or weaves, create a robust yet lightweight framework that can be strategically designed to accommodate embedded electronic components 110 while maintaining structural integrity of the wearable ring device 104. This layered architecture (e.g., architecture including multiple carbon fiber layers 114) enables the integration of conductive traces, sensors, and other electronic elements between or within the carbon fiber layers 114, effectively utilizing the material not just as a housing but as an active component of the device’s electrical and sensing systems.

For example, as shown in FIG. 1, the ring-shaped housing 106 may be formed by creating/forming a first carbon fiber layer 114-a, and disposing a conductive material 116 over a surface of the first carbon fiber layer 114-a. In some aspects, the conductive material 116 may be used to form one or more conductive traces 118 (e.g., electrical traces, electrical vias). The conductive traces 118 embedded within or between the carbon fiber layers 114 may be configured to electrically couple the various electronic components 110, including any batteries, processors, sensors, and communication components of the wearable ring device 104.

The conductive traces 118 may be formed through various methods, including but not limited to, direct printing, metal/chemical deposition, etching, aerosol jet printing, or selective manipulation of the carbon fiber material itself. Additionally, the conductive material 116 may be strategically placed within the ring-shaped housing 106 to create electrical pathways, antennas, or other functional elements. In this example, an additional carbon fiber layer 114 (as shown and described in FIG. 2) may be formed over the conductive traces 118, thereby embedding (e.g., “weaving”) the conductive traces 118 within/between the carbon fiber layers 114. Additionally, or alternatively, the electronic components 110 may be embedded (at least partially) within the carbon fiber layers 114, such that the electronic components 110 are disposed (at least partially) within the carbon fiber layers 114. In some cases, the ring-shaped housing 106 may include multiple “layers” of conductive traces 118 that are interleaved with carbon fiber layers 114. The different “layers” of conductive traces may be coupled with one another via conductive vias, thereby creating a “network” of conductive traces that are interwoven throughout the wearable ring device 104.

The electrical properties of carbon fiber materials may be particularly advantageous in the context of wearable ring devices 104. Carbon fibers possess inherent electrical conductivity that can be selectively utilized through careful control of fiber orientation and matrix composition. This property may enable the creation of integrated antenna structures, electromagnetic shields, and conductive pathways within the ring housing itself, reducing the need for additional components and conserving valuable space within the wearable ring device 104.

In some aspects, the carbon fiber layers 114 may be formed to create both conductive and non-conductive regions within the ring-shaped housing 106, which may allow for sophisticated electrode configurations and sensor integration without compromising the device’s mechanical properties (as shown and described in FIG. 2). Further, carbon fiber layers 114 may be used to create “windows” of non-conductive material to facilitate wireless communications, where the “windows” are integrated seamlessly into the form factor of the device, and which may be imperceptible from a visual standpoint. The non-conductive windows may be created by selectively orienting carbon fibers in specific regions to minimize electrical conductivity, or by incorporating non-conductive matrix materials in designated areas during the manufacturing process.

The manufacturing flexibility of carbon fiber composites (e.g., carbon fiber layers 114) may provide additional advantages for the wearable ring device 104. Through various processing techniques such as layup, molding, and selective resin infiltration, the material properties of the ring-shaped housing 106 (e.g., carbon fiber layers 114) can be precisely tailored to specific regions of the device. For example, areas requiring greater flexibility for user comfort can be engineered with different fiber orientations or resin systems as compared to regions requiring maximum rigidity for component protection. For instance, the wearable ring device 104 may be constructed such that the inner curved surface (and inner layers) are flexible, whereas the outer curved surface (e.g., outer layers) are more rigid for durability. By way of another example, a first radial span of the wearable ring device (such as a span that includes a battery or other electrical components) may be manufactured to be more rigid to provide support and protection for such components, where a second radial span of the wearable device may be manufactured to be softer or more flexible for improved user comfort. This ability to create functionally-graded properties within a single structure may enable optimization of both user comfort and device performance. In particular, previous approaches that have attempted to combine flexible and non-flexible (e.g., rigid) materials have encountered difficulties with the interface between the different materials, as it may be difficult to retain the bond between the different materials at the interface. However, this problem may be addressed using the techniques described herein, as the different carbon fiber layers 114 may be formed/selected with varying properties (e.g., varying levels of flexibility), thereby avoiding the issue of different materials being joined at some interface.

Additionally, as will be further shown and described with reference to FIG. 2, in some cases, the carbon fiber layers 114 may be formed/processed in a flat state, and subsequently formed into the ring-shaped housing 106, simplifying the integration of electronic components and reducing manufacturing complexity of the wearable ring device 104.

The carbon fiber materials of the carbon fiber layers 114 may also maintain their properties across a wide temperature range and resist degradation from exposure to typical environmental conditions encountered during daily wear. When properly engineered with appropriate polymer matrices, carbon fiber composites may provide effective environmental protection for sensitive electronic components, while maintaining the necessary biocompatibility for prolonged skin contact. These characteristics, combined with the ability to precisely machine and finish such carbon fiber materials, may enable the creation of durable, reliable wearable devices that can withstand the demands of continuous use while maintaining their functional properties and aesthetic appearance.

Accordingly, in accordance with aspects of the present disclosure, the wearable ring device 104 may be manufactured with CFRPs in order to combine the structural advantages of carbon fibers with carefully selected polymer matrices to create a multifunctional material platform for the wearable ring device 104. The polymer matrix, which may include specialized thermoset or thermoplastic resins, may be used to bind the carbon fibers together (within and across the respective carbon fiber layers 114), as well as protect the electronic components 110 of the wearable ring device 104. That is, the wearable ring device 104 may be constructed of carbon fiber materials that are interwoven or impregnated with other materials, such as thermoplastics, silicon, graphene, etc. In the context of the wearable ring device 104, CFRPs can be engineered to incorporate varying degrees of flexibility, electrical conductivity, and thermal management properties through selective matrix composition and fiber orientation, enabling the creation of devices that optimize both performance and user comfort.

The integration of the electronic components 110 within CFRP structures (e.g., carbon fiber layers 114) may present unique advantages for the wearable ring device 104. By utilizing specific matrix formulations and processing techniques, the electronic components 110 (e.g., LEDs, photodetectors, other sensors, processors, and communication components) can be directly embedded within the CFRP structure (e.g., carbon fiber layers 114) during manufacture. This approach eliminates the need for separate component housings, and may reduce overall device complexity. For example, by embedding/weaving the electronic components 110 directly into the CFRP structure (e.g., carbon fiber layers 114), the need for a separate PCB for mounting the electronic components 110 may be eliminated, thereby enabling the wearable ring device 104 to be made thinner and lighter (due at least partly to the omission of the PCB). Furthermore, the polymer matrix of the CFRP structure (e.g., polymer matrix of the carbon fiber layers 114 forming the ring-shaped housing 106) may be selectively modified to create regions with varying electrical properties, allowing for the integration of antennas, electrodes, and other functional elements directly within the composite structure while maintaining necessary electrical isolation between components.

The composite nature of CFRPs may also enable the integration of additional functional materials within the structure of the wearable ring device 104 (e.g., within the ring-shaped housing). For example, nanoparticles (e.g., semiconductor particles such as quantum dots), conductive fillers, optical fibers, and/or specialized additives can be incorporated into the polymer matrix to enhance specific properties or add new functionalities. For example, the inclusion of ferromagnetic materials (e.g., magnetic materials) can enable inductive charging capabilities, while the addition of specialized particles can improve thermal management or provide electromagnetic shielding. These modifications can be selectively applied to specific regions of the wearable ring device 104 through careful control of the matrix composition and processing parameters.

In some aspects, the fibers of the carbon fiber layers 114 may be doped with ferromagnetic materials themselves. Additionally, or alternatively, ferromagnetic materials may be sandwiched between or among the carbon fiber layers 114 (e.g., magnetic layer between two carbon fiber layers 114). The inclusion of magnetic materials within the carbon fiber layers 114, and/or inclusion of magnetic layers themselves, may be used to achieve several benefits or characteristics. First, the magnetic materials/layers may serve to shield the internal electronics from magnetic fields, which may be particularly useful to protect memory devices or chips that use magnetic memory (MRAM). Additionally, the inclusion of magnetic materials may be used to add a slightly magnetic region to the ring for mechanical alignment to either a manufacturing fixture, a user device, and/or a charger. For example, the carbon fiber layers 114 around a portion of the ring may be “doped” with magnetic materials such that the portion of the ring can magnetically interact with magnets of a charging device in order to rotate or otherwise align the ring on the charging device in a position that facilitates charging (e.g., magnetic portion of the ring interacts with magnets of the charger to align charging components of the ring with charging components of the charger). or a user device like a charger. Additionally, the inclusion of magnetic materials/layers in the ring may be used to facilitate or improve performance of a magnetic/coil antenna within the ring.

By way of another example, in some cases, optical fibers may be embedded within and interwoven throughout the carbon fiber layers 114 of the device, similar to how the conductive traces 118 may be embedded/interwoven within the carbon fiber material. In some cases, optical fibers may run to the exterior surfaces of the wearable ring device 104 (e.g., outer and inner curved/circumferential surfaces, lateral/side surfaces) such that the optical fibers may be used to transmit and/or receive light exterior to the wearable ring device 104. For instance, optical fibers may be routed to the inner curved surface of the wearable ring device 104 (e.g., distributed around the inner curved/circumferential surface) to be used as light-emitting components for collecting PPG and other physiological data. In such cases, laser diodes may be used to couple light into the optical fibers that will be output by the optical fibers. By way of another example, optical fibers may be routed to the outer curved surface to perform various functions, including displaying information to the user (e.g., battery status information, a “measurement mode,” user feedback, ring orientation/rotation information, etc.), gesture recognition/ring-inputted commands, and/or determining an orientation/rotation of the ring.

For instance, optical fibers on the outer surface of the wearable ring device 104 may be used to collect light, and can therefore be used to determine where on the outer surface of the ring is being touched by the user or another object (e.g., where optical fibers are not receiving light, or receiving less light). As such, light collected by the optical fibers may be used to determine the orientation/rotation of the ring on the user’s finger (e.g., by assuming that the side of the ring on the dorsal side of the user’s hand receives more light than the palm-side of the hand), determine which finger the ring is being worn on (e.g., by identifying which sides of the ring are receiving less light, and therefore are likely facing adjacent fingers), determine gestures performed by the user (e.g., user tapping/covering different portions of the ring), etc. Such information may be used to selectively activate/deactivate sensors on the ring, perform various actions (e.g., “tag” an event, cause an external device to perform an action, etc.), determine an identity of the user (e.g., authentication for payments and access), and the like.

In some cases, optical fibers embedded or interwoven into/across the carbon fiber layers 114 may be used as a “strain gauge” to measure pressure around various portions of the wearable ring device 104 (e.g., measure skin contact pressure, measure pressure exerted by the user on the outer surface of the ring for gesture recognition, etc.). For instance, pressure exerted against the optical fibers may reduce (or eliminate) the amount of light that is able to pass through the optical fibers. As such, by measuring the amount of light that passes through optical fibers, the wearable ring device 104 may be able to estimate the amount of pressure exerted at various parts of the ring.

In other cases, optical fibers embedded or interwoven into/across the carbon fiber layers 114 may be used to facilitate authentication, payments, and other use-cases. For example, in cases where wearable ring devices 104 include optical fibers that extend to the outer surface of the rings, two users may be able to touch their rings together, where the optical fibers may be used to communicate light (e.g., via predetermined light pulses, predetermined pulsing patterns) between the respective rings in order to authorize a payment from one user to the other. By way of another example, the wearable ring device 104 may be configured to authenticate an identity of the user (e.g., via collected physiological data, detected gestures, etc.), where the ring uses the optical fibers to transmit light to the exterior of the ring to validate the user’s identity and enable the user to unlock a door, access their vehicle, etc.

In additional or alternative implementations, the wearable ring device 104 may include microfluidic channels that are embedded (e.g., interwoven) throughout the carbon fiber matrix (e.g., within/between the carbon fiber layers 114), similar to how the conductive traces 118 and/or optical fibers may be embedded/interwoven throughout the device. In such cases, the microfluidic channels may be used to move fluid throughout the ring, which may be used for drug delivery (e.g., microdosing), fluid analytics (e.g., sweat/blood analysis), and the like. For example, in some cases, the wearable ring device 104 may include small needles or other structures that are used to collect fluids (e.g., blood, sweat, etc.) from the user, and direct the fluids to the microfluidic channels. In such cases, the wearable ring device 104 may be configured to analyze the fluids, such as by transmitting light through the microfluidic channels and measuring light that is reflected/absorbed by the fluid. Such techniques may be used to perform blood glucose measurements (e.g., continuous glucose monitoring (CGM)), determine hydration levels, analyze concentrations of substances within the user’s blood (e.g., blood alcohol, etc.), and the like.

By way of another example, the wearable ring device 104 may include semiconductor particles (e.g., quantum dots) that are embedded within the carbon fiber layers 114. The semiconductor particles may be connected to one another (and other components of the ring, such as a battery) via the conductive traces 118 and/or conductive vias. In some cases, semiconductor particles may be used to generate light to alter the visual appearance (e.g., color) of the ring, and/or to convey information to a user (e.g., via flashing different color lights, and/or by using semiconductor particles to create some sort of display/GUI on the surface of the ring). In some cases, the power required to change a color (e.g., change an activation state) of a semiconductor particle may be relatively high, whereas the power required to maintain a semiconductor particle at a specific color (e.g., maintain the activation state associated with a specific color) may be relatively small. As such, in some cases, the user may be able to change the color of the ring (e.g., change the color/activation states of the semiconductor particles) by placing the wearable ring device 104 on the charger (where the charger can provide the power used to change the color/activation states). Subsequently, the battery of the wearable ring device 104 may be used to maintain the current/power required to maintain the color/activation state of the semiconductor particles through the use of a small amount of current provided to the semiconductor particles (e.g., via conductive carbon fibers of the carbon fiber layers 114).

FIG. 2 shows an example of a manufacturing diagram 200 that supports a wearable device with carbon fiber components in accordance with aspects of the present disclosure. Aspects of the manufacturing diagram 200 may implement, or be implemented by, aspects of the wearable ring device 104 shown and described in FIG. 1. In particular, the manufacturing diagram 200 shown and described in FIG. 2 illustrates different components and features of the ring-shaped housing 106 shown and described in FIG. 1.

As noted previously herein, the ring-shaped housing 106 may include a 3D layered composite structure formed from CFRP materials. For example, referring to the top portion of FIG. 2, the ring-shaped housing 106 may include a first carbon fiber layer 114-a and a second carbon fiber layer 114-b, which together form a layered composite structure. While only two carbon fiber layers 114 are shown and described in FIG. 2, it is noted herein that a wearable device may be manufactured with any number/quantity of carbon fiber layers 114. This configuration enables the integration of various functional elements (e.g., conductive traces 118) between and within the carbon fiber layers 114, while maintaining the structural integrity of the device. For instance, the ring-shaped housing 106 may include multiple “layers” of conductive traces 118 that are “sandwiched” (e.g., embedded, interwoven) between respective carbon fiber layers 114. The first carbon fiber layer 114-a and the conductive traces 118 shown and described in FIG. 2 may be examples of the corresponding components shown and described in FIG. 1.

In some aspects, the conductive traces 118 may include or be used as antennas for facilitating wireless communication between the wearable ring device 104 and external devices, such as user devices. In other cases, the carbon fiber material (e.g., carbon fiber layers 114) themselves may be used as antenna components of the ring.

In some aspects, the composite structure of the ring-shaped housing 106 may be formed to create both conductive and non-conductive regions within the ring-shaped housing 106, which may allow for sophisticated electrode configurations and sensor integration without compromising the device’s mechanical properties (as shown and described in FIG. 2). For example, as shown in FIG. 2, the ring-shaped housing 106 may include a first electrode 202-a and a second electrode 202-b. In some cases, the electrodes 202 may be formed seamlessly into the structure of the ring-shaped housing 106 (e.g., without dedicated conductive “pads”) through careful selection of the material properties embedded/woven into the carbon fiber layers 114. For example, the quantity or density of the conductive traces 118 may be selectively manufactured/controlled around different portions of the wearable ring device in order to create or control electrical properties across the respective portions/regions of the wearable ring device 104. For instance, the electrodes 202 may be formed using the conductive traces 118 embedded within the carbon fiber layers 114 (e.g., higher density or quantity of electrical traces 118 may be used to form the electrodes 202).

In some aspects, the electrodes 202 may be formed on the inner surface and/or outer surface (e.g., inner/outer curved surface, inner/outer circumferential surface) of the ring-shaped housing 106 to contact the user’s finger tissue for various physiological measurements, including ECG and bioimpedance measurements. For example, electrodes 202 formed on the inner and/or outer curved surface of the wearable ring device 104 may be used for capacitive touch sensing capabilities, enabling processors of the wearable device to determine where the user’s tissue is contacting the wearable ring device 104. Such capacitive touch sensing capabilities may be used to determine the fit or orientation (e.g., rotation) of the wearable ring device, identify gestures performed by the user (e.g., user touching specific portions of the ring to perform various actions), determine an identity of the user (e.g., authentication for payments or access by comparing ECG measurements to a user’s ECG profile), or any combination thereof.

In some cases, as shown in the top portion of FIG. 2, the composite structure of the ring-shaped housing 106 (e.g., carbon fiber layers 114) may be first formed in a flat/linear orientation that can be subsequently curved into the ring-shaped housing 106. The ability to process these materials in a flat orientation before final curving/forming may allow for more precise component placement and the creation of complex internal architectures, such as the conductive traces 118. Stated differently, electronic components 110 of the wearable ring device 104 may be embedded within (e.g., woven into) and/or attached to the carbon fiber layers 114 while in the flat orientation (thereby enabling more precise placement), then curved into the ring form factor.

In some aspects, various matrix systems for the composite structure of the ring-shaped housing (e.g., thermoplastic polyurethanes (TPUs), specialized epoxy resins, Kevlar, etc.) may be used to achieve specific mechanical and electrical properties in different regions of the wearable ring device 104. Stated differently, materials with different properties may be embedded, woven, impregnated, or otherwise included within the various carbon fiber layers 114 with varying quantities/densities at different portions of the ring-shaped housing 106 to achieve various characteristics/properties. For example, in some cases, different carbon fiber layers 114 may be woven or otherwise formed to exhibit varying rigidity and/or flexibility, which may enable wearable ring devices 104 to be created with flexible/pliable inner curved surfaces for improved user comfort, and harder/rigid outer curved surfaces to protect the components of the wearable device. Additionally, the polymer matrix can be engineered to provide specific surface properties, such as biocompatibility for skin contact or enhanced durability for external surfaces.

For instance, in some cases, the carbon fiber layers 114 may be specifically formed or selected to enable the ring to “break” off from the user’s finger when a threshold force is exerted on the ring (e.g., “breakaway” ring to prevent damage to the user’s finger). In other cases, the carbon fiber matrix may be formed to improve the fit to the user’s finger, such as by enabling the ring-shaped housing 106 to change shape/size based on an application of heat (e.g., body heat from the user’s tissue) and/or electrical current (e.g., current generated by the battery of the ring and distributed throughout the ring via the conductive traces 118). Additionally, or alternatively, the carbon fiber matrix may be embedded or impregnated with shape memory alloys.

In some aspects, the conductive traces 118 may be coupled with one another using vias, a PCB, a printed wiring board (PWB), or other electrical means. For example, referring to the bottom portion of FIG. 2, the ring-shaped housing 106 may include a PCB 204 that is used to connect the various conductive traces 118. In effect, this creates multiple “loops” of conductive traces 118 that extend around a circumference of the wearable ring device 104 (e.g., extend 360° around the ring). The bottom portion of FIG. 2 may illustrate a portion of the ring-shaped housing 106 after curving from the linear architecture shown in the top portion of FIG. 2. In other cases, the PCB 204 may extend radially around at least a portion of the wearable ring device 104, where PCB 204 may be integrated within (e.g., disposed within or between) the carbon fiber layers 114, electrical traces 118, etc.

In some aspects, processors of the wearable ring device 104 may select which “loops” (or other “paths” or subsets) of conductive traces 118 may be used or activated at a time to perform various functions, such as performing wireless communications, charging, etc. For example, the outermost “loops” of conductive traces (e.g., loops closest to the lateral sides of the ring and/or loops closest to the inner and/or outer curved surfaces of the ring) may be used/activated for wireless charging (e.g., resonant charging, inductive charging) for the wearable ring device 104. In other cases, various subsets of conductive traces may be used/activated for the processors of the wearable device to communicate with various components (e.g., electronic components 110, electrodes 202, radio frequency circuitry) of the ring.

In some aspects, the PCB 204 may be integrated within the ring-shaped housing 106 and positioned between the first carbon fiber layer 114-a and the second carbon fiber layer 114-b. In other cases, the PCB 204 (or some other collection of electrical vias, etc.) may span a height of multiple carbon fiber layers 114 in order to connect multiple “layers” of conductive traces 118. The PCB 204 may include or support various electronic components 110, and may provide connections between the respective conductive traces 118 that extend through/between the carbon fiber layers 114. In this regard, the conductive traces 118 may form an interconnected electrical network throughout the wearable ring device 104, enabling electrical communication between components while utilizing the carbon fiber structure as both a housing and a functional element of the device’s electrical system. For instance, the PCB 204 (or other suitable electrical structure/component) may be configured to selectively “connect” various loops of conductive traces 118 with one another, as well as connect various conductive traces within different “layers” of the ring (e.g., connect a conductive trace 118 positioned proximate to the outer curved surface of the ring to a conductive trace 118 positioned proximate to the inner curved surface of the ring).

The layered construction of the wearable ring device 104/ring-shaped housing 106 illustrated in FIG. 2 demonstrates how the carbon fiber materials can be utilized to create a sophisticated, integrated device structure. The arrangement allows for the embedding of various components, including optical fibers, semiconductor particles, and ferromagnetic materials, while maintaining a compact form factor. The respective carbon fiber layers 114 may be specifically engineered to provide environmental protection, electromagnetic shielding, or enhanced antenna functionality, depending on the specific requirements of the device. This multi-layer approach enables the creation of a highly integrated wearable device that maximizes functionality while minimizing size and complexity.

While aspects of the present disclosure are primarily described in the context of finger-worn wearable ring devices 104, it is to be understood that the techniques described herein may also be applied to other form factors of wearable devices, such as wrist-worn devices (e.g., smart watches, smart bracelets), chest straps, etc.

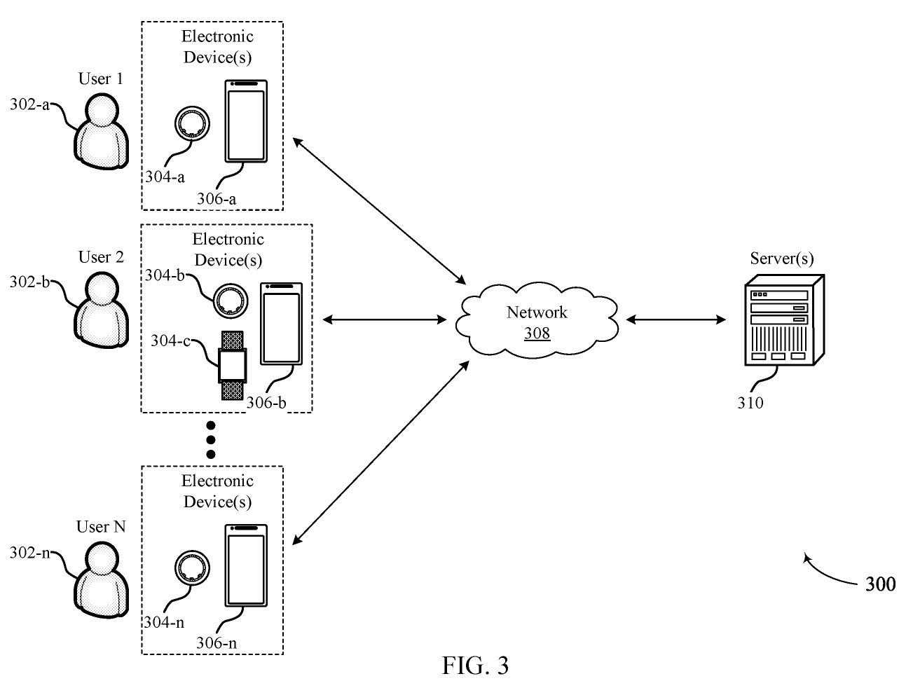

FIG. 3 illustrates an example of a system 300 that supports wearable devices with carbon fiber components in accordance with aspects of the present disclosure. The system 300 includes a plurality of electronic devices (e.g., wearable devices 304, user devices 306) that may be worn and/or operated by one or more users 302. The system 300 further includes a network 308 and one or more servers 310.

The electronic devices may include any electronic devices known in the art, including wearable devices 304 (e.g., ring wearable devices, watch wearable devices, etc.), user devices 306 (e.g., smartphones, laptops, tablets). The electronic devices associated with the respective users 302 may include one or more of the following functionalities: 1) measuring physiological data, 2) storing the measured data, 3) processing the data, 4) providing outputs (e.g., via GUIs) to a user 302 based on the processed data, and 5) communicating data with one another and/or other computing devices. Different electronic devices may perform one or more of the functionalities.

Example wearable devices 304 may include wearable computing devices, such as a ring computing device (hereinafter “ring”) configured to be worn on a user’s 302 finger, a wrist computing device (e.g., a smart watch, fitness band, or bracelet) configured to be worn on a user’s 302 wrist, and/or a head mounted computing device (e.g., glasses/goggles). Wearable devices 304 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 304 may also be attached to, or included in, articles of clothing. For example, wearable devices 304 may be included in pockets and/or pouches on clothing. As another example, wearable device 304 may be clipped and/or pinned to clothing, or may otherwise be maintained within the vicinity of the user 302. Example articles of clothing may include, but are not limited to, hats, shirts, gloves, pants, socks, outerwear (e.g., jackets), and undergarments. In some implementations, wearable devices 304 may be included with other types of devices such as training/sporting devices that are used during physical activity. For example, wearable devices 304 may be attached to, or included in, a bicycle, skis, a tennis racket, a golf club, and/or training weights.

Much of the present disclosure may be described in the context of a wearable device 304, which may include finger-worn wearable devices, wrist-worn wearable devices, and the like. Accordingly, the terms “wearable device 304,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 306 may include handheld mobile computing devices, such as smartphones and tablet computing devices. User devices 306 may also include personal computers, such as laptop and desktop computing devices. Other example user devices 306 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 306 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 304, user devices 306) may measure physiological parameters of respective users 302, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 304), mobile device application, or a server computing device may process received physiological data that was measured by other devices.

In some implementations, a user 302 may operate, or may be associated with, multiple electronic devices, some of which may measure physiological parameters and some of which may process the measured physiological parameters. In some implementations, a user 302 may have a ring (e.g., wearable device 304) that measures physiological parameters. The user 302 may also have, or be associated with, a user device 306 (e.g., mobile device, smartphone), where the wearable device 304 and the user device 306 are communicatively coupled to one another. In some cases, the user device 306 may receive data from the wearable device 304 and perform some/all of the calculations described herein. In some implementations, the user device 306 may also measure physiological parameters described herein, such as motion/activity parameters.

For example, as illustrated in FIG. 3, a first user 302-a (User 1) may operate, or may be associated with, a wearable device 304-a (e.g., wearable ring device) and a user device 306-a that may operate as described herein. In this example, the user device 306-a associated with user 302-a may process/store physiological parameters measured by the wearable device 304-a. Comparatively, a second user 302-b (User 2) may be associated with wearable devices 304-b and 304-c (e.g., wearable ring device and a wrist-worn wearable device, such as a watch) and a user device 306-b, where the user device 306-b associated with user 302-b may process/store physiological parameters measured by the wearable devices 304-b and 304-c. Moreover, an nth user 302-n (User N) may be associated with an arrangement of electronic devices described herein (e.g., wearable device 304-n, user device 306-n). In some aspects, wearable devices 304 (e.g., wearable ring devices, wrist-worn wearable devices) and other electronic devices may be communicatively coupled to the user devices 306 of the respective users 302 via Bluetooth, Wi-Fi, and other wireless protocols. Moreover, in some cases, the wearable device 304 and the user device 306 may be included within (or make up) the same device. For example, in some cases, the wearable device 304 may be configured to execute an application associated with the wearable device 304, and may be configured to display data via a GUI.

In some implementations, the wearable devices 304 (e.g., wearable ring devices) of the system 300 may be configured to collect physiological data from the respective users 302 based on arterial blood flow within the user’s finger. In particular, a wearable ring device may utilize one or more light-emitting components, such as LEDs (e.g., red LEDs, green LEDs) that emit light on the palm-side of a user’s finger to collect physiological data based on arterial blood flow within the user’s finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 300 may be configured to collect physiological data from the respective users 302 based on blood flow diffused into a microvascular bed of skin with capillaries and arterioles. For example, the system 300 may collect PPG data based on a measured amount of blood diffused into the microvascular system of capillaries and arterioles. In some implementations, the wearable device 304 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 304 (e.g., around an inner surface of the wearable ring device) has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 300 (e.g., user devices 306, wearable devices 304) may be communicatively coupled to one or more servers 310 via wired or wireless communication protocols. For example, as shown in FIG. 3, the electronic devices (e.g., user devices 306) may be communicatively coupled to one or more servers 310 via a network 308. The network 308 may implement transfer control protocol and internet protocol (TCP/IP), such as the Internet, or may implement other network 308 protocols. Network connections between the network 308 and the respective electronic devices may facilitate transport of data via email, web, text messages, mail, or any other appropriate form of interaction within a computer network 308. For example, in some implementations, the wearable device 304-a associated with the first user 302-a may be communicatively coupled to the user device 306-a, where the user device 306-a is communicatively coupled to the servers 310 via the network 308. In additional or alternative cases, wearable devices 304 (e.g., wearable ring devices, wrist-worn wearable devices such as watches) may be directly communicatively coupled to the network 308.

The system 300 may offer an on-demand database service between the user devices 306 and the one or more servers 310. In some cases, the servers 310 may receive data from the user devices 306 via the network 308, and may store and analyze the data. Similarly, the servers 310 may provide data to the user devices 306 via the network 308. In some cases, the servers 310 may be located at one or more data centers. The servers 310 may be used for data storage, management, and processing. In some implementations, the servers 310 may provide a web-based interface to the user device 306 via web browsers.

In some aspects, the system 300 may detect periods of time that a user 302 is asleep, and classify periods of time that the user 302 is asleep into one or more sleep stages (e.g., sleep stage classification). For example, as shown in FIG. 3, User 302-a may be associated with a wearable device 304-a (e.g., wearable ring device) and a user device 306-a. In this example, the wearable device 304-a may collect physiological data associated with the user 302-a, including temperature, heart rate, HRV, respiratory rate, and the like. In some aspects, data collected by the wearable device 304-a may be input to a machine learning classifier, where the machine learning classifier is configured to determine periods of time that the user 302-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 302-a via a GUI of the user device 306-a. Sleep stage classification may be used to provide feedback to a user 302-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 300 may utilize circadian rhythm-derived features to further improve physiological data collection, data processing procedures, and other techniques described herein. The term circadian rhythm may refer to a natural, internal process that regulates an individual’s sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 302-a via the wearable device 304-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 302 to generate tailored, individualized circadian rhythm adjustment models that are specific to each respective user 302.

In some aspects, the system 300 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 300 may support techniques for wearable ring devices with carbon fiber materials, as described previously herein.

It should be appreciated by a person skilled in the art that one or more aspects of the disclosure may be implemented in a system 300 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

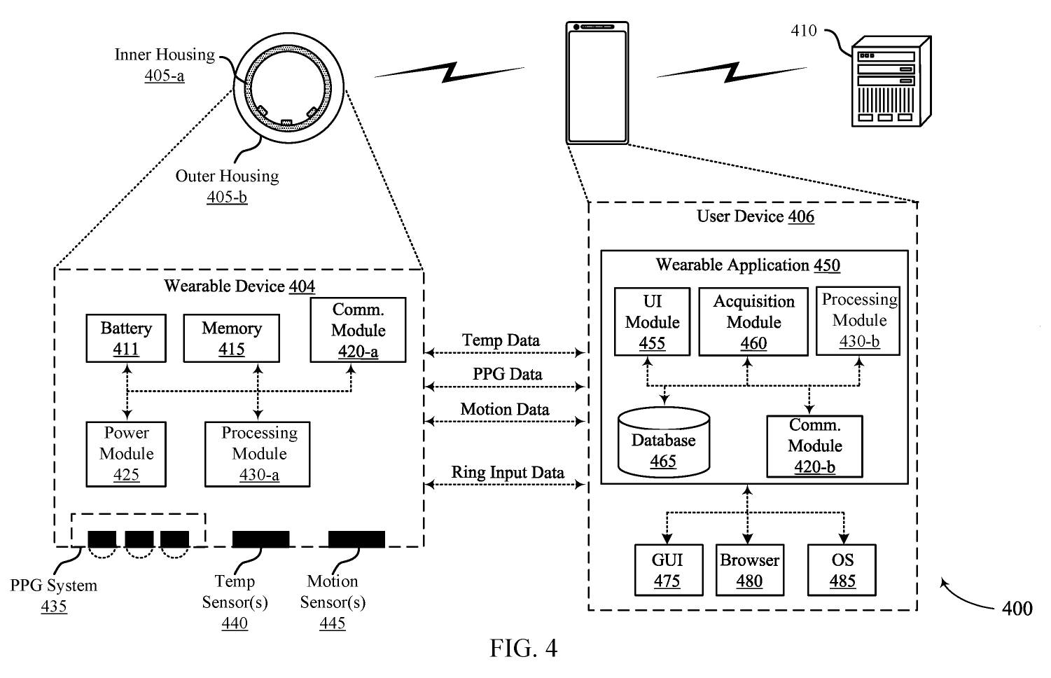

FIG. 4 illustrates an example of a system 400 that supports wearable devices with carbon fiber components in accordance with aspects of the present disclosure. The system 400 may implement, or be implemented by, system 300. In particular, system 400 illustrates a wearable device 404 (e.g., wearable ring device), a user device 406, and a server 410, as described with reference to FIG. 3.

In some aspects, the wearable device 404 (e.g., wearable ring device) may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 400 further includes a user device 406 (e.g., a smartphone) in communication with the wearable device 404. For example, the wearable device 404 may be in wireless and/or wired communication with the user device 406. In some implementations, the wearable device 404 may send measured and processed data (e.g., temperature data, photoplethysmogram (PPG) data, motion/accelerometer data, ring input data, and the like) to the user device 406. The user device 406 may also send data to the wearable device 404, such as firmware/configuration updates. The user device 406 may process data. In some implementations, the user device 406 may transmit data to the server 410 for processing and/or storage.

The wearable device 404 may include a housing 405 that may include an inner housing 405-a and an outer housing 405-b. In some aspects, the inner housing 405-a, the outer housing 405-b, or both, may include a curved profile/surface. In particular, the housing 405 may exhibit any curved or “circumferential” profile, including a circular profile, an elliptical profile, and the like. Moreover, in some cases, the inner housing 405-a, the outer housing 405-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 405 of the wearable device 404 may store or otherwise include various components of the ring including, but not limited to, device electronics, a power source (e.g., battery 411, and/or capacitor), one or more substrates (e.g., printable circuit boards) that interconnect the device electronics and/or power source, and the like. The device electronics may include device modules (e.g., hardware/software), such as: a processing module 430-a, a memory 415, a communication module 420-a, a power module 425, and the like. The device electronics may also include one or more sensors. Example sensors may include one or more temperature sensors 440, a PPG sensor assembly (e.g., PPG system 435), and one or more motion sensors 445.

The sensors may include associated modules (not illustrated) configured to communicate with the respective components/modules of the wearable device 404, and generate signals associated with the respective sensors. In some aspects, each of the components/modules of the wearable device 404 may be communicatively coupled to one another via wired or wireless connections. Moreover, the wearable device 404 may include additional and/or alternative sensors or other components that are configured to collect physiological data from the user, including light sensors (e.g., LEDs), oximeters, and the like.

The wearable device 404 shown and described with reference to FIG. 4 is provided solely for illustrative purposes. As such, the wearable device 404 may include additional or alternative components as those illustrated in FIG. 4. Additional or alternative wearable devices 404 that provide functionality described herein may be fabricated. For example, wearable devices 404 with fewer components (e.g., sensors) may be fabricated. In a specific example, a wearable device 404 with a single temperature sensor 440 (or other sensor), a power source, and device electronics configured to read the single temperature sensor 440 (or other sensor) may be fabricated. In another specific example, a temperature sensor 440 (or other sensor) may be attached to a user’s finger (e.g., using adhesives, wraps, clamps, spring loaded clamps, etc.). In this case, the sensor may be wired to another computing device, such as a wrist worn computing device that reads the temperature sensor 440 (or other sensor). In other examples, a wearable device 404 that includes additional sensors and processing functionality may be fabricated.

The housing 405 may include one or more housing components. The housing 405 may include an outer housing 405-b component (e.g., a shell) and an inner housing 405-a component (e.g., a molding). The housing 405 may include additional components (e.g., additional layers) not explicitly illustrated in FIG. 4. For example, in some implementations, the wearable device 404 may include one or more insulating layers that electrically insulate the device electronics and other conductive materials (e.g., electrical traces) from the outer housing 405-b. The housing 405 may provide structural support for the device electronics, battery 411, substrate(s), and other components. For example, the housing 405 may protect the device electronics, battery 411, and substrate(s) from mechanical forces, such as pressure and impacts. The housing 405 may also protect the device electronics, battery 411, and substrate(s) from water and/or other chemicals.

The inner housing 405-a may be configured to interface with the user’s finger. The inner housing 405-a may be formed from a polymer (e.g., a medical grade polymer) or other material. In some implementations, the inner housing 405-a may be transparent. For example, the inner housing 405-a may be transparent to light emitted by the PPG LEDs. In some implementations, the inner housing 405-a component may be molded onto the outer housing 405-b. For example, the inner housing 405-a may include a polymer that is molded (e.g., injection molded) to fit into an outer housing 405-b metallic shell.

The inner housing 405-a and the outer housing 405-b may be fabricated from one or more materials. In some implementations, the inner housing 405-a, the outer housing 405-b, or both, may include a metal, such as titanium, that may provide strength and abrasion resistance at a relatively light weight. Additionally, or alternatively, the inner housing 405-a, and/or the outer housing 405-b may also be fabricated from other materials, such polymers, plastic materials, epoxy materials, ceramic materials, and the like. In some implementations, the outer housing 405-b may be protective as well as decorative.

The wearable device 404 may include one or more substrates (not illustrated). The device electronics and battery 411 may be included on the one or more substrates. For example, the device electronics and battery 411 may be mounted on one or more substrates. Example substrates may include one or more printed circuit boards (PCBs), such as flexible PCB (e.g., polyimide). In some implementations, the electronics/battery 411 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 411 to the device electronics.

The device electronics, battery 411, and substrates may be arranged in the wearable device 404 in a variety of ways. In some implementations, one substrate that includes device electronics may be mounted along the bottom of the wearable device 404 (e.g., the bottom half), such that the sensors (e.g., PPG system 435, temperature sensors 440, motion sensors 445, and other sensors) interface with the underside of the user’s finger. In these implementations, the battery 411 may be included along the top portion of the wearable device 404 (e.g., on another substrate).

The various components/modules of the wearable device 404 represent functionality (e.g., circuits and other components) that may be included in the wearable device 404. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 415 (memory module) of the wearable device 404 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 415 may store any of the data described herein. For example, the memory 415 may be configured to store data (e.g., motion data, temperature data, PPG data) collected by the respective sensors and PPG system 435. Furthermore, memory 415 may include instructions that, when executed by one or more processing circuits, cause the modules to perform various functions attributed to the modules herein. The device electronics of the wearable device 404 described herein are only example device electronics. As such, the types of electronic components used to implement the device electronics may vary based on design considerations.

The functions attributed to the modules of the wearable device 404 (e.g., wearable ring device) described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 430-a of the wearable device 404 may include one or more processors (e.g., processing units), microcontrollers, digital signal processors, systems on a chip (SOCs), and/or other processing devices. The processing module 430-a communicates with the modules included in the wearable device 404. For example, the processing module 430-a may transmit/receive data to/from the modules and other components of the wearable device 404, such as the sensors. As described herein, the modules may be implemented by various circuit components. Accordingly, the modules may also be referred to as circuits (e.g., a communication circuit and power circuit).

The processing module 430-a may communicate with the memory 415. The memory 415 may include computer-readable instructions that, when executed by the processing module 430-a, cause the processing module 430-a to perform the various functions attributed to the processing module 430-a herein. In some implementations, the processing module 430-a (e.g., a microcontroller) may include additional features associated with other modules, such as communication functionality provided by the communication module 420-a (e.g., an integrated Bluetooth Low Energy transceiver) and/or additional onboard memory 415.

The communication module 420-a may include circuits that provide wireless and/or wired communication with the user device 406 (e.g., communication module 420-b of the user device 406). In some implementations, the communication modules 420-a, 420-b may include wireless communication circuits, such as Bluetooth circuits and/or Wi-Fi circuits. In some implementations, the communication modules 420-a, 420-b can include wired communication circuits, such as Universal Serial Bus (USB) communication circuits. Using the communication module 420-a, the wearable device 404 and the user device 406 may be configured to communicate with each other. The processing module 430-a of the ring may be configured to transmit/receive data to/from the user device 406 via the communication module 420-a. Example data may include, but is not limited to, motion data, temperature data, pulse waveforms, heart rate data, HRV data, PPG data, and status updates (e.g., charging status, battery charge level, and/or wearable device 404 configuration settings). The processing module 430-a of the ring may also be configured to receive updates (e.g., software/firmware updates) and data from the user device 406.

The wearable device 404 may include a battery 411 (e.g., a rechargeable battery 411). An example battery 411 may include a Lithium-Ion or Lithium-Polymer type battery 411, although a variety of battery 411 options are possible. The battery 411 may be wirelessly charged. In some implementations, the wearable device 404 may include a power source other than the battery 411, such as a capacitor. The power source (e.g., battery 411 or capacitor) may have a curved geometry that matches the curve of the wearable device 404. In some aspects, a charger or other power source may include additional sensors that may be used to collect data in addition to, or that supplements, data collected by the wearable device 404 itself. Moreover, a charger or other power source for the wearable device 404 may function as a user device 406, in which case the charger or other power source for the wearable device 404 may be configured to receive data from the wearable device 404, store and/or process data received from the wearable device 404, and communicate data between the wearable device 404 and the servers 410.

In some aspects, the wearable device 404 includes a power module 425 that may control charging of the battery 411. For example, the power module 425 may interface with an external wireless charger that charges the battery 411 when interfaced with the wearable device 404. The charger may include a datum structure that mates with a wearable device 404 datum structure to create a specified orientation with the wearable device 404 during charging. The power module 425 may also regulate voltage(s) of the device electronics, regulate power output to the device electronics, and monitor the state of charge of the battery 411. In some implementations, the battery 411 may include a protection circuit module (PCM) that protects the battery 411 from high current discharge, over voltage during charging, and under voltage during discharge. The power module 425 may also include electro-static discharge (ESD) protection.

The one or more temperature sensors 440 may be electrically coupled to the processing module 430-a. The temperature sensor 440 may be configured to generate a temperature signal (e.g., temperature data) that indicates a temperature read or sensed by the temperature sensor 440. The processing module 430-a may determine a temperature of the user in the location of the temperature sensor 440. For example, in the wearable device 404, temperature data generated by the temperature sensor 440 may indicate a temperature of a user at the user’s finger (e.g., skin temperature). In some implementations, the temperature sensor 440 may contact the user’s skin. In other implementations, a portion of the housing 405 (e.g., the inner housing 405-a) may form a barrier (e.g., a thin, thermally conductive barrier) between the temperature sensor 440 and the user’s skin. In some implementations, portions of the wearable device 404 configured to contact the user’s finger may have thermally conductive portions and thermally insulative portions. The thermally conductive portions may conduct heat from the user’s finger to the temperature sensors 440. The thermally insulative portions may insulate portions of the wearable device 404 (e.g., the temperature sensor 440) from ambient temperature.

In some implementations, the temperature sensor 440 may generate a digital signal (e.g., temperature data) that the processing module 430-a may use to determine the temperature. As another example, in cases where the temperature sensor 440 includes a passive sensor, the processing module 430-a (or a temperature sensor 440 module) may measure a current/voltage generated by the temperature sensor 440 and determine the temperature based on the measured current/voltage. Example temperature sensors 440 may include a thermistor, such as a negative temperature coefficient (NTC) thermistor, or other types of sensors including resistors, transistors, diodes, and/or other electrical/electronic components.

The processing module 430-a may sample the user’s temperature over time. For example, the processing module 430-a may sample the user’s temperature according to a sampling rate. An example sampling rate may include one sample per second, although the processing module 430-a may be configured to sample the temperature signal at other sampling rates that are higher or lower than one sample per second. In some implementations, the processing module 430-a may sample the user’s temperature continuously throughout the day and night. Sampling at a sufficient rate (e.g., one sample per second) throughout the day may provide sufficient temperature data for analysis described herein.

The processing module 430-a may store the sampled temperature data in memory 415. In some implementations, the processing module 430-a may process the sampled temperature data. For example, the processing module 430-a may determine average temperature values over a period of time. In one example, the processing module 430-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 415 may store the average temperature values over time. In some implementations, the memory 415 may store average temperatures (e.g., one per minute) instead of sampled temperatures in order to conserve memory 415.

The sampling rate, which may be stored in memory 415, may be configurable. In some implementations, the sampling rate may be the same throughout the day and night. In other implementations, the sampling rate may be changed throughout the day/night. In some implementations, the wearable device 404 may filter/reject temperature readings, such as large spikes in temperature that are not indicative of physiological changes (e.g., a temperature spike from a hot shower). In some implementations, the wearable device 404 may filter/reject temperature readings that may not be reliable due to other factors, such as excessive motion during exercise (e.g., as indicated by a motion sensor 445).

The wearable device 404 (e.g., communication module) may transmit the sampled and/or average temperature data to the user device 406 for storage and/or further processing. The user device 406 may transfer the sampled and/or average temperature data to the server 410 for storage and/or further processing.

Although the wearable device 404 is illustrated as including a single temperature sensor 440, the wearable device 404 may include multiple temperature sensors 440 in one or more locations, such as arranged along the inner housing 405-a near the user’s finger. In some implementations, the temperature sensors 440 may be stand-alone temperature sensors 440. Additionally, or alternatively, one or more temperature sensors 440 may be included with other components (e.g., packaged with other components), such as with the accelerometer and/or processor.

The processing module 430-a may acquire and process data from multiple temperature sensors 440 in a similar manner described with respect to a single temperature sensor 440. For example, the processing module 430 may individually sample, average, and store temperature data from each of the multiple temperature sensors 440. In other examples, the processing module 430-a may sample the sensors at different rates and average/store different values for the different sensors. In some implementations, the processing module 430-a may be configured to determine a single temperature based on the average of two or more temperatures determined by two or more temperature sensors 440 in different locations on the finger.

The temperature sensors 440 on the wearable device 404 (e.g., wearable ring device) may acquire distal temperatures at the user’s finger (e.g., any finger). For example, one or more temperature sensors 440 on the wearable device 404 may acquire a user’s temperature from the underside of a finger or at a different location on the finger. In some implementations, the wearable device 404 may continuously acquire distal temperature (e.g., at a sampling rate). Although distal temperature measured by a wearable device 404 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the wearable device 404 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 404 may include a PPG system 435. The PPG system 435 may include one or more optical transmitters that transmit light. The PPG system 435 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 435 may indicate the perfusion of blood in the illuminated region. For example, the PPG signal may indicate blood volume changes in the illuminated region caused by a user’s pulse pressure. The processing module 430-a may sample the PPG signal and determine a user’s pulse waveform based on the PPG signal. The processing module 430-a may determine a variety of physiological parameters based on the user’s pulse waveform, such as a user’s respiratory rate, heart rate, HRV, oxygen saturation, and other circulatory parameters.

In some implementations, the PPG system 435 may be configured as a reflective PPG system 435 where the optical receiver(s) receive transmitted light that is reflected through the region of the user’s finger. In some implementations, the PPG system 435 may be configured as a transmissive PPG system 435 where the optical transmitter(s) and optical receiver(s) are arranged opposite to one another, such that light is transmitted directly through a portion of the user’s finger to the optical receiver(s).

The number and ratio of transmitters and receivers included in the PPG system 435 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 435.

The PPG system 435 illustrated in FIG. 4 may include a reflective PPG system 435 in some implementations. In these implementations, the PPG system 435 may include a centrally located optical receiver (e.g., at the bottom of the wearable device 404) and two optical transmitters located on each side of the optical receiver. In this implementation, the PPG system 435 (e.g., optical receiver) may generate the PPG signal based on light received from one or both of the optical transmitters. In other implementations, other placements, combinations, and/or configurations of one or more optical transmitters and/or optical receivers are contemplated.

The processing module 430-a may control one or both of the optical transmitters to transmit light while sampling the PPG signal generated by the optical receiver. In some implementations, the processing module 430-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 435 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 430-a may store the pulse waveform in memory 415 in some implementations. The processing module 430-a may process the pulse waveform as it is generated and/or from memory 415 to determine user physiological parameters described herein.

The processing module 430-a may determine the user’s heart rate based on the pulse waveform. For example, the processing module 430-a may determine heart rate (e.g., in beats per minute) based on the time between peaks in the pulse waveform. The time between peaks may be referred to as an interbeat interval (IBI). The processing module 430-a may store the determined heart rate values and IBI values in memory 415.

The processing module 430-a may determine HRV over time. For example, the processing module 430-a may determine HRV based on the variation in the IBIs. The processing module 430-a may store the HRV values over time in the memory 415. Moreover, the processing module 430-a may determine the user’s respiratory rate over time. For example, the processing module 430-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 430-a may store user respiratory rate values over time in the memory 415.

The wearable device 404 may include one or more motion sensors 445, such as one or more accelerometers (e.g., 6-D accelerometers) and/or one or more gyroscopes (gyros). The motion sensors 445 may generate motion signals that indicate motion of the sensors. For example, the wearable device 404 may include one or more accelerometers that generate acceleration signals that indicate acceleration of the accelerometers. As another example, the wearable device 404 may include one or more gyro sensors that generate gyro signals that indicate angular motion (e.g., angular velocity) and/or changes in orientation. The motion sensors 445 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 430-a may sample the motion signals at a sampling rate (e.g., 50Hz) and determine the motion of the wearable device 404 based on the sampled motion signals. For example, the processing module 430-a may sample acceleration signals to determine acceleration of the wearable device 404. As another example, the processing module 430-a may sample a gyro signal to determine angular motion. In some implementations, the processing module 430-a may store motion data in memory 415. Motion data may include sampled motion data as well as motion data that is calculated based on the sampled motion signals (e.g., acceleration and angular values).

The wearable device 404 may store a variety of data described herein. For example, the wearable device 404 may store temperature data, such as raw sampled temperature data and calculated temperature data (e.g., average temperatures). As another example, wearable device 404 may store PPG signal data, such as pulse waveforms and data calculated based on the pulse waveforms (e.g., heart rate values, IBI values, HRV values, and respiratory rate values). The wearable device 404 may also store motion data, such as sampled motion data that indicates linear and angular motion.

The wearable device 404, or other computing device, may calculate and store additional values based on the sampled/calculated physiological data. For example, the processing module 430 may calculate and store various metrics, such as sleep metrics (e.g., a Sleep Score), activity metrics, and readiness metrics. In some implementations, additional values/metrics may be referred to as “derived values.” The wearable device 404, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 404 is oriented on the user’s finger and if the wearable device 404 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 430-a may compress the data stored in memory 415. For example, the processing module 430-a may delete sampled data after making calculations based on the sampled data. As another example, the processing module 430-a may average data over longer periods of time in order to reduce the number of stored values. In a specific example, if average temperatures for a user over one minute are stored in memory 415, the processing module 430-a may calculate average temperatures over a five minute time period for storage, and then subsequently erase the one minute average temperature data. The processing module 430-a may compress data based on a variety of factors, such as the total amount of used/available memory 415 and/or an elapsed time since the wearable device 404 last transmitted the data to the user device 406.

Although a user’s physiological parameters may be measured by sensors included on a wearable device 404, other devices may measure a user’s physiological parameters. For example, although a user’s temperature may be measured by a temperature sensor 440 included in a wearable device 404, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 404 can make physiological measurements in a resting/sleep state in order to acquire cleaner physiological signals. In one example, the wearable device 404 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 404 may be configured to collect, store, and/or process data, and may transfer any of the data described herein to the user device 406 for storage and/or processing. In some aspects, the user device 406 includes a wearable application 450, an operating system 485 (OS), a web browser application (e.g., web browser 480), one or more additional applications, and a GUI 475. The user device 406 may further include other modules and components, including sensors, audio devices, haptic feedback devices, and the like. The wearable application 450 may include an example of an application (e.g., “app”) that may be installed on the user device 406. The wearable application 450 may be configured to acquire data from the wearable device 404, store the acquired data, and process the acquired data as described herein. For example, the wearable application 450 may include a user interface (UI) module 455, an acquisition module 460, a processing module 430-b, a communication module 420-b, and a storage module (e.g., database 465) configured to store application data.

In some cases, the wearable device 404 and the user device 406 may be included within (or make up) the same device. For example, in some cases, the wearable device 404 may be configured to execute the wearable application 450, and may be configured to display data via the GUI 475.

The various data processing operations described herein may be performed by the wearable device 404, the user device 406, the servers 410, or any combination thereof. For example, in some cases, data collected by the wearable device 404 may be pre-processed and transmitted to the user device 406. In this example, the user device 406 may perform some data processing operations on the received data, may transmit the data to the servers 410 for data processing, or both. For instance, in some cases, the user device 406 may perform processing operations that require relatively low processing power and/or operations that require a relatively low latency, whereas the user device 406 may transmit the data to the servers 410 for processing operations that require relatively high processing power and/or operations that may allow relatively higher latency.

In some aspects, the wearable device 404 (e.g., wearable ring device), user device 406, and server 410 of the system 400 may be configured to evaluate sleep patterns for a user. In particular, the respective components of the system 400 may be used to collect data from a user via the wearable device 404, and generate one or more scores (e.g., Sleep Score, Readiness Score) for the user based on the collected data. For example, as noted previously herein, the wearable device 404 of the system 400 may be worn by a user to collect data from the user, including temperature, heart rate, HRV, and the like. Data collected by the wearable device 404 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 404 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 400 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7–9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5°C higher than the highest temperature during the longest period. In some aspects, the ring may measure a user’s body temperature while the user is asleep, and the system 400 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

As described previously herein, in some implementations, the wearable device 404 may be constructed (at least partially) using carbon fiber materials (e.g., CFRP materials). For example, as described previously herein, the inner housing 405-a, the outer housing 405-b, or both, may include one or more carbon fiber layers 114 that are embedded, interweaved, or impregnated with various electronic components 110 of the device.

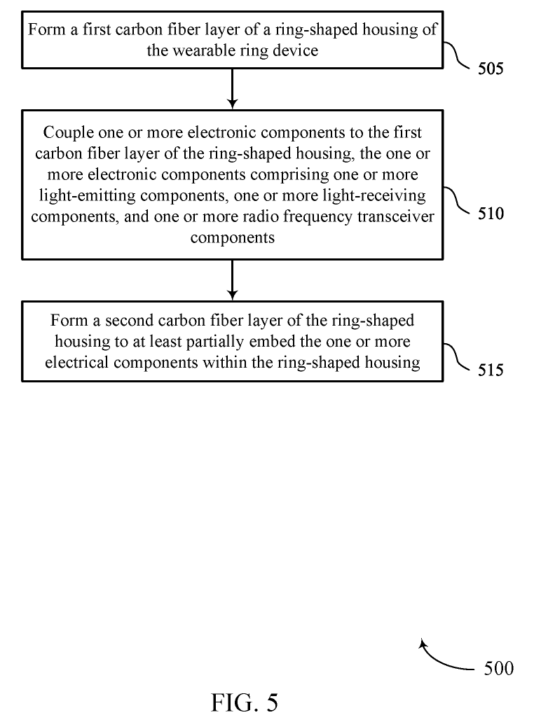

FIG. 5 shows a flowchart illustrating a method 500 that supports a method for manufacturing a wearable device with carbon fiber components in accordance with aspects of the present disclosure. The operations of the method 500 may be implemented by one or more manufacturing components (e.g., fabrication machines, etc.).

At 505, the method may include forming a first carbon fiber layer of a ring-shaped housing of the wearable ring device. The operations of 505 may be performed in accordance with examples as disclosed herein.

At 510, the method may include coupling one or more electronic components to the first carbon fiber layer of the ring-shaped housing, the one or more electronic components comprising one or more light-emitting components, one or more light-receiving components, and one or more radio frequency transceiver components. The operations of 510 may be performed in accordance with examples as disclosed herein.

At 515, the method may include forming a second carbon fiber layer of the ring-shaped housing to at least partially embed the one or more electronic components within the ring-shaped housing. The operations of 515 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:

Aspect 1: A wearable ring device, comprising: a ring-shaped housing comprising one or more carbon fiber layers, the ring-shaped housing configured to at least partially surround a finger of a user; one or more electronic components embedded within, or attached to, the one or more carbon fiber layers of the ring-shaped housing, the one or more electronic components comprising one or more light-emitting components, one or more light-receiving components, and one or more radio frequency transceiver components; a battery electrically coupled with the one or more electronic components; and one or more processors configured to: acquire physiological data associated with the user based at least in part on light transmitted via the one or more light-emitting components and received by the one or more light-receiving components; and transmit the physiological data to a user device via the one or more radio frequency transceiver components.

Aspect 2: The wearable ring device of aspect 1, wherein the ring-shaped housing comprises: a plurality of conductive traces embedded within the one or more carbon fiber layers, wherein the plurality of conductive traces are configured to electrically couple at least the one or more electronic components, the battery, and the one or more processors.

Aspect 3: The wearable ring device of aspect 2, wherein the plurality of conductive traces extend radially around a full circumference of the wearable ring device.

Aspect 4: The wearable ring device of any of aspects 1 through 3, wherein the ring-shaped housing comprises an inner curved surface of the wearable ring device, an outer curved surface of the wearable ring device, or both, the inner curved surface configured to at least partially contact a tissue of the user, the wearable ring device further comprising: a first electrode disposed at least partially within the inner curved surface; and a second electrode disposed at least partially within the inner curved surface or the outer curved surface, wherein the first electrode and the second electrode are formed via a plurality of conductive traces embedded within the one or more carbon fiber layers.

Aspect 5: The wearable ring device of aspect 4, wherein the one or more processors are configured to: cause the first electrode to generate an electrical current; and perform one or more electrocardiogram measurements, bioimpedance measurements, or both, based at least in part on the electrical current received through the tissue of the user via the second electrode.

Aspect 6: The wearable ring device of any of aspects 4 through 5, wherein the one or more processors are configured to: perform a capacitive touch sensing procedure based at least in part on a contact between the tissue of the user and the first electrode, the second electrode or both; and determine an orientation of the wearable ring device on the finger of the user, a gesture performed by the user, an identity of the user, or any combination thereof, based at least in part on the capacitive touch sensing procedure.

Aspect 7: The wearable ring device of any of aspects 1 through 6, wherein the ring-shaped housing comprises: a plurality of optical fibers embedded within the one or more carbon fiber layers, wherein the one or more light-emitting components, one or more light-receiving components, or both, comprise the plurality of optical fibers.

Aspect 8: The wearable ring device of any of aspects 1 through 7, further comprising: a plurality of semiconductor particles embedded within the one or more carbon fiber layers.

Aspect 9: The wearable ring device of aspect 8, wherein the plurality of semiconductor particles are electrically coupled with the battery, the plurality of semiconductor particles are configured to generate light in response to an electrical current received from the battery.

Aspect 10: The wearable ring device of any of aspects 1 through 9, wherein the one or more carbon fiber layers comprise at least a portion of an antenna associated with the one or more radio frequency transceiver components.

Aspect 11: The wearable ring device of any of aspects 1 through 10, further comprising: an inductive charging mechanism configured to recharge the battery, wherein the inductive charging mechanism comprises a plurality of ferromagnetic materials embedded within the one or more carbon fiber layers.

Aspect 12: The wearable ring device of any of aspects 1 through 11, wherein the ring-shaped housing comprises an outer curved surface of the wearable ring device.

Aspect 13: The wearable ring device of any of aspects 1 through 12, wherein the ring-shaped housing comprises an inner curved surface of the wearable ring device, the inner curved surface is configured to at least partially contact a tissue of the user.

Aspect 14: A method for manufacturing a wearable ring device, comprising: forming a first carbon fiber layer of a ring-shaped housing of the wearable ring device, coupling one or more electronic components to the first carbon fiber layer of the ring-shaped housing, the one or more electronic components comprising one or more light-emitting components, one or more light-receiving components, and one or more radio frequency transceiver components; and forming a second carbon fiber layer of the ring-shaped housing to at least partially embed the one or more electronic components within the ring-shaped housing.

Aspect 15: The method of aspect 14, further comprising: disposing the ring-shaped housing within a mold subsequent to forming the second carbon fiber layer; and forming a molded material on a surface of the ring-shaped housing based at least in part on disposing the ring-shaped housing within the mold, wherein the molded material forms an inner curved surface of the wearable ring device, an outer curved surface of the wearable ring device, or both.

Aspect 16: The method of any of aspects 14 through 15, further comprising: forming a plurality of conductive traces on the first carbon fiber layer, wherein the plurality of conductive traces are configured to electrically couple at least the one or more electronic components, a battery, and one or more processors of the wearable ring device, wherein the plurality of conductive traces are at least partially embedded within the ring-shaped housing based at least in part on forming the second carbon fiber layer.

Aspect 17: The method of aspect 16, wherein the plurality of conductive traces extend radially around a full circumference of the wearable ring device.

Aspect 18: The method of any of aspects 14 through 17, further comprising: forming a plurality of optical fibers on the first carbon fiber layer, wherein the one or more light-emitting components, one or more light-receiving components, or both, comprise the plurality of optical fibers, and wherein the plurality of optical fibers are at least partially embedded within the ring-shaped housing based at least in part on forming the second carbon fiber layer.

Aspect 19: The method of any of aspects 14 through 18, further comprising: embedding a plurality of semiconductor particles within the first carbon fiber layer, the second carbon fiber layer, or both.

Aspect 20: The method of aspect 19, further comprising: forming a plurality of conductive traces on or within the first carbon fiber layer, the second carbon fiber layer, or both, wherein the plurality of semiconductor particles are electrically coupled with a battery of the wearable ring device via the plurality of conductive traces.

Aspect 21: The method of manufacturing of any of aspects 14 through 20 for manufacturing the wearable ring device of any of aspects 1 through 13.

Aspect 22: A wearable ring device manufactured by the method of any of aspects 14 through 20.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
a ring-shaped housing comprising one or more carbon fiber layers, the ring-shaped housing configured to at least partially surround a finger of a user;
one or more electronic components embedded within, or attached to, the one or more carbon fiber layers of the ring-shaped housing, the one or more electronic components comprising one or more light-emitting components, one or more light-receiving components, and one or more radio frequency transceiver components;
a battery electrically coupled with the one or more electronic components; and
one or more processors configured to:
acquire physiological data associated with the user based at least in part on light transmitted via the one or more light-emitting components and received by the one or more light-receiving components; and
transmit the physiological data to a user device via the one or more radio frequency transceiver components.

2. The wearable ring device of claim 1, wherein the ring-shaped housing comprises:
a plurality of conductive traces embedded within the one or more carbon fiber layers, wherein the plurality of conductive traces are configured to electrically couple at least the one or more electronic components, the battery, and the one or more processors.

3. The wearable ring device of claim 2, wherein the plurality of conductive traces extend radially around a full circumference of the wearable ring device.

4. The wearable ring device of claim 1, wherein the ring-shaped housing comprises an inner curved surface of the wearable ring device, an outer curved surface of the wearable ring device, or both, the inner curved surface configured to at least partially contact a tissue of the user, the wearable ring device further comprising:
a first electrode disposed at least partially within the inner curved surface; and
a second electrode disposed at least partially within the inner curved surface or the outer curved surface, wherein the first electrode and the second electrode are formed via a plurality of conductive traces embedded within the one or more carbon fiber layers.

5. The wearable ring device of claim 4, wherein the one or more processors are configured to:
cause the first electrode to generate an electrical current; and
perform one or more electrocardiogram measurements, bioimpedance measurements, or both, based at least in part on the electrical current received through the tissue of the user via the second electrode.

6. The wearable ring device of claim 4, wherein the one or more processors are configured to:
perform a capacitive touch sensing procedure based at least in part on a contact between the tissue of the user and the first electrode, the second electrode, or both; and
determine an orientation of the wearable ring device on the finger of the user, a gesture performed by the user, an identity of the user, or any combination thereof, based at least in part on the capacitive touch sensing procedure.

7. The wearable ring device of claim 1, wherein the ring-shaped housing comprises:
a plurality of optical fibers embedded within the one or more carbon fiber layers, wherein the one or more light-emitting components, the one or more light-receiving components, or both, comprise the plurality of optical fibers.

8. The wearable ring device of claim 1, further comprising:
a plurality of semiconductor particles embedded within the one or more carbon fiber layers.

9. The wearable ring device of claim 8, wherein the plurality of semiconductor particles are electrically coupled with the battery, wherein the plurality of semiconductor particles are configured to generate light in response to an electrical current generated by the battery.

10. The wearable ring device of claim 1, wherein the one or more carbon fiber layers comprise at least a portion of an antenna associated with the one or more radio frequency transceiver components.

11. The wearable ring device of claim 1, further comprising:
an inductive charging mechanism configured to recharge the battery, wherein the inductive charging mechanism comprises a plurality of ferromagnetic materials embedded within the one or more carbon fiber layers.

12. The wearable ring device of claim 1, wherein the ring-shaped housing comprises an outer curved surface of the wearable ring device.

13. The wearable ring device of claim 1, wherein the ring-shaped housing comprises an inner curved surface of the wearable ring device, wherein the inner curved surface is configured to at least partially contact a tissue of the user.

14. A method for manufacturing a wearable ring device, comprising:
forming a first carbon fiber layer of a ring-shaped housing of the wearable ring device,
coupling one or more electronic components to the first carbon fiber layer of the ring-shaped housing, the one or more electronic components comprising one or more light-emitting components, one or more light-receiving components, and one or more radio frequency transceiver components; and
forming a second carbon fiber layer of the ring-shaped housing to at least partially embed the one or more electronic components within the ring-shaped housing.

15. The method of claim 14, further comprising:
disposing the ring-shaped housing within a mold subsequent to forming the second carbon fiber layer; and
forming a molded material on a surface of the ring-shaped housing based at least in part on disposing the ring-shaped housing within the mold, wherein the molded material forms an inner curved surface of the wearable ring device, an outer curved surface of the wearable ring device, or both.

16. The method of claim 14, further comprising:
forming a plurality of conductive traces on or within the first carbon fiber layer, wherein the plurality of conductive traces are configured to electrically couple at least the one or more electronic components, a battery, and one or more processors of the wearable ring device, wherein the plurality of conductive traces are at least partially embedded within the ring-shaped housing based at least in part on forming the second carbon fiber layer.

17. The method of claim 16, wherein the plurality of conductive traces extend radially around a full circumference of the wearable ring device.

18. The method of claim 14, further comprising:
forming a plurality of optical fibers on the first carbon fiber layer, wherein the one or more light-emitting components, the one or more light-receiving components, or both, comprise the plurality of optical fibers, and wherein the plurality of optical fibers are at least partially embedded within the ring-shaped housing based at least in part on forming the second carbon fiber layer.

19. The method of claim 14, further comprising:
embedding a plurality of semiconductor particles within the first carbon fiber layer, the second carbon fiber layer, or both.

20. The method of claim 19, further comprising:
forming a plurality of conductive traces on or within the first carbon fiber layer, the second carbon fiber layer, or both, wherein the plurality of semiconductor particles are electrically coupled with a battery of the wearable ring device via the plurality of conductive traces.
